(19) United States
(12) Patent Application Publication
(10) Pub. No.: US 2012/0294952 A1
(43) Pub. Date: Nov. 22, 2012

(54) ANTITUSSIVE COMPOSITIONS AND METHODS

(75) Inventors: Zak Zarbock, South Jordan, UT (US); Bryce Johnson, South Jordan, UT (US); G. Daniel Villareal, South Jordan, UT (US)

(73) Assignee: Zarbees, Inc., South Jordan, UT (US)

(21) Appl. No.: 13/475,433

(22) Filed: May 18, 2012

Related U.S. Application Data
(60) Provisional application No. 61/487,589, filed on May 18, 2011.

(51) Int. Cl. A61K 35/64; A61P 11/14

(52) U.S. Cl. 424/537

(57) ABSTRACT

Compositions and methods for providing relief from coughing are provided.

### TECHNICAL FIELD

This invention relates to antitussive compositions and to methods of their use in patients suffering from coughing.

### SUMMARY

The present invention relates to antitussive compositions for providing relief from coughing, including night coughing, and to methods of providing relief to patients suffering from coughing, including night coughing by administering such compositions.

In one aspect, the invention relates to antitussive compositions comprising honey and a sleep aid such as melatonin. In some embodiments, the antitussive composition further comprises an orally acceptable carrier, wherein the composition is substantially in one of the following forms: an aqueous-based liquid, or a form dissolvable in an aqueous-based liquid, such as, but not limited to the following: a syrup, a suspension, a spray, a lozenge, a chewable lozenge, a powder, a tablet, a quick-dissolve powder, a quick-dissolve tablet, a quick-dissolve strip, or a chewable tablet.

In some of these embodiments, the composition comprises melatonin at a concentration from about 0.0007% (w/w) to about 0.5% (w/w). In some of these embodiments, the remainder of the composition is honey. In others, some lesser percentage is honey, with the remainder being made up of other constituent substances.

In other embodiments, the composition is provided in a dosage unit form. In some of these, each dosage unit comprises from about 0.1 mg to about 20 mg melatonin. In some of these, the remainder is honey. In others, other compounds supplement the honey.

In some embodiments the honey used is granular honey. In other such embodiments, the honey is granulated buckwheat honey.

The antitussive compositions of the invention may also optionally further comprise one or more additional components, including, without limitation, fruit, vegetables, herbs, or berries, or extracts thereof. In some embodiments, the antitussive composition comprises elderberry extract. In some embodiments, the antitussive composition comprises grape skin extract. In some embodiments, the antitussive composition comprises grape seed extract. In others, the antitussive composition comprises green tea extract.

The antitussive compositions of the invention may also optionally further comprise one or more vitamins, including vitamins added to the compositions. In some such embodiments, the antitussive composition further comprises added B-vitamins. In other embodiments, the antitussive composition further comprises added vitamin C. In another embodiment, the antitussive composition further comprises added vitamin D. In a further embodiment, any combination of vitamins, including those listed herein, may be envisioned.

The antitussive compositions provided herein may also optionally further comprise minerals, including minerals added to the compositions. In some of these, the antitussive composition comprises added zinc.

In another aspect, the invention relates to methods of providing relief from coughing to an individual. In one embodiment, the method comprises the step of administering to the individual an effective amount of the antitussive compositions comprising honey and melatonin provided herein.

In a related embodiment, the step of administering an effective amount of the antitussive composition to the individual results in the individual receiving from about 0.1 mg to about 20 mg of melatonin.

In another embodiment, the coughing is night coughing, and the step of administering an effective amount of the antitussive composition to the individual is performed just prior to the individual retiring for sleep.

In another embodiment, the antitussive composition is administered perorally. In a related embodiment, the antitussive composition is in powder form, and prior to the step of administering the antitussive composition, the antitussive composition is dissolved in an aqueous-based liquid.

Additional aspects and advantages will be apparent from the following detailed description of preferred embodiments, which proceeds with reference to the accompanying drawings.

### DETAILED DESCRIPTION OF PREFERRED EMBODIMENTS

The disclosure herein is directed toward providing antitussive compositions and methods for providing relief from coughing to an individual.

It will be readily understood that the embodiments, as generally described herein, are exemplary. The following more detailed description of various embodiments is not intended to limit the scope of the present disclosure, but is merely representative of various embodiments. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified.

### Definitions

Unless specifically defined otherwise, the technical terms, as used herein, have their normal meaning as understood in the art. The following terms are specifically defined with examples for the sake of clarity.

“Antitussive” is used to describe a composition or method which can be used to provide relief from coughing to an individual. Relief from coughing may be in terms of decreased frequency of coughing fits, decreased severity of coughing fits, or decreased discomfort related to coughing.

“Aqueous-based liquid” means a liquid in which the most abundant molecule is H₂O. Aqueous-based liquids include water, solutions such as juices or other drinks for human consumption. When administered as a tablet, quick-dissolve tablet or in a similar such form, biological secretions, such as saliva, would be the aqueous-based liquid.

“Dosage unit form” refers to a composition intended for metered or dosed human consumption where the composition is provided in individual units which comprise one or more integer values of a dose. Compositions in dosage unit form include individual lozenges, tablets, or other such similar suitable forms which provide one or more doses of the composition. Compositions in dosage unit form also include powders packaged in premeasured doses.

“Vitamin” means an organic compound required in small amounts by an organism. In humans, vitamins include vitamin A, the B-vitamins (B₁, B₂, B₃, B₅, B₆, B₇, B₉, B₁₂), vitamin C, vitamin D, vitamin E and vitamin K.

“Added” vitamins or minerals refer to purified vitamins or minerals supplemented to a composition to fortify the composition.

“Sleep aid” refers to particular compounds or extracts that aid in the onset of sleep and/or relieve stress. Such compounds or extracts include: 5-hydroxytryptophan (5-HTP), gamma-aminobutyric acid (GABA), glycine, L-theanine, L-tryptophan, melatonin, chamomile flower, lemon balm, passion flower, and valerian. Other similar compounds may also be suitable for use in the present disclosure. This term includes the compounds used singly, and in combination.

“Honey” refers to a material that is produced and collected from bees. Honey types may sometimes be classified by which flower(s) it is produced from. Several examples of which are buckwheat, clover, dandelion, honeydew, or wildflower honey. Other honey types exist and may be suitable for use in the composition. These types of honey may be used singly, or may also be blended to form a honey blend that is then used in the composition.

### Compositions

In one aspect, the invention relates to antitussive compositions. In a general embodiment, the antitussive compositions comprise honey and a sleep aid, such as, in some specific compositions, melatonin.

The honey may be in any of its commercially available forms, and of any type. For example, the honey may be comb honey, chunk honey, raw honey, pasteurized honey, strained honey, ultrafiltered honey, ultrasonicated honey, whipped honey, crystallized honey, dried honey, granulated honey or powdered honey. In some embodiments, the honey is buckwheat honey. In some of these, the honey is granulated buckwheat honey.

Likewise, the sleep aid may be obtained commercially. The sleep aid may be in solution in an aqueous-based liquid, or it may be provided in a crystalline or a powdered form. In specific embodiments, the sleep aid is melatonin, and may be obtained commercially, and may also be in solution in an aqueous-based liquid, or it may be provided in a crystalline or a powdered form.

In some embodiments, the honey and the at least one sleep aid, such as melatonin, are mixed together to form the antitussive composition. For example, in some embodiments, powdered honey or granulated honey are mixed with crystalline melatonin or powdered melatonin to yield an antitussive composition.

In certain embodiments, the antitussive composition also comprises an orally acceptable carrier. Orally acceptable carriers are well known in the art, and a skilled person would be able to choose the correct orally acceptable carrier for a composition depending on the application.

Orally acceptable carriers come in many forms including water, aqueous-based liquids, syrups, suspensions, sprays, lozenges, chewable lozenges, powders, tablets quick-dissolve powders, quick-dissolve strips, quick-dissolve tablets and chewable tablets. In certain applications, different carrier forms will be more appropriate. For example in certain embodiments, the antitussive composition is formulated for children, and comprises an orally acceptable carrier in the form of a chewable lozenge, or a chewable tablet. In other embodiments, the antitussive composition is formulated for people with difficulty swallowing, and comprises an orally acceptable carrier in the form of a spray.

In some embodiments, the composition is substantially in a powder form. In some of these embodiments, the composition is provided in a powdered or granulated form for reconstitution in water or an aqueous-based liquid.

In certain embodiments, the antitussive composition comprises an amount of honey and a sleep aid such as, in some specific embodiments, melatonin, effective to imbue the composition with antitussive properties. In some embodiments, the composition comprises melatonin in a concentration from about 0.0007% (w/w) to about 0.5% (w/w). In related embodiments, the composition comprises melatonin in a concentration from about 0.007% (w/w) to about 0.07% (w/w). In related embodiments, the composition comprises melatonin in a concentration from about 0.02% (w/w) to about 0.04% (w/w).

In some embodiments, the composition is provided in a dosage unit form. In related embodiments, the composition is provided in a dosage unit form such that each dose provides from about 0.1 mg to about 70 mg melatonin. In related embodiments, each dose provides from about 1 mg to about 10 mg melatonin. In related embodiments, each dose provides from about 3 to about 5 mg melatonin.

In certain embodiments, the composition may also comprise additional components, including, without limitation, fruit, vegetables, herbs, or berries, or extracts thereof. In some embodiments, the antitussive composition comprises elderberry extract. In some embodiments, the antitussive composition comprises grape skin extract. In some embodiments, the antitussive composition comprises grape seed extract. In others, the antitussive composition comprises green tea extract. In another embodiment, the anti-tussive composition may comprise a combination of one or more of the above components, such as combining grape seed extract with elderberry extract, or combining elderberry extract with grape seed extract and grape skin extract, or the like.

The extracts may be purchased commercially, or they may be prepared by extraction methods such as those known to those of ordinary skill in the art. In certain embodiments, extracts of grape are included in the composition. In a related embodiment, the composition comprises grape seed extract. In another related embodiment, the composition comprises grape skin extract. In a further related embodiment, all, or any combination of the various grape extracts may be combined in the same composition.

In certain embodiments, the composition comprises green tea extract. In certain embodiments, the composition comprises elderberry extract.

In certain embodiments, the composition is fortified with vitamins or minerals. Vitamins and minerals are well known in the art, and the skilled person can assess the correct dosage for vitamins and minerals for a particular application. For example the skilled person may use the recommended daily allowance (RDA) values for determining the doses of vitamins and minerals.

In some embodiments, the composition comprises added vitamins. In a related embodiment, the composition comprises added vitamin C. In a related embodiment, the composition comprises one or more added B-vitamins. In a related embodiment, the composition comprises added vitamin D.

In certain embodiments, the composition comprises added minerals. For example in one embodiment, the composition comprises added zinc.

In order to make the composition more palatable for certain individuals, the composition may also comprise natural or artificial sweeteners, natural or artificial colors, or natural or artificial flavors.

In order to help induce sleep, a sleep aid or a mixture of sleep aids may be added to the composition. One embodiment may have any one of the following sleep aids in the composition: 5-HTP, chamomile flower, GABA, glycine, lemon balm, L-theanine, L-tryptophan, melatonin, passion flower, and valerian. A second embodiment may have any number of those sleep aids listed combined in a composition.

### Methods

In another aspect, the invention relates to methods of providing relief from coughing to an individual.

In one general embodiment, the method comprises administering to the individual an effective amount of a composition comprising honey and sleep aid, such as, in some embodiments, melatonin. In certain embodiments, one or more of the embodiments of antitussive compositions disclosed herein are administered to the individual.

The methods may be used to treat any variety of cough. For example the methods may be used to treat coughing in children, coughing in adults, daytime coughing or nighttime coughing. In certain embodiments, the method is utilized to provide relief from nighttime coughing. In a related embodiment, the step of administering the composition is performed just prior to the individual retiring for sleep. In some of these, the composition is administered between 10 minutes and 2 hours prior to sleep. In others, the composition is administered between 10 and 30 minutes prior to sleep. In related embodiments, the step of administering the composition is performed 1, 2, 3, 4, 5, 10, 20, 30, 60, 90, 120, or more than 120 minutes prior to the individual retiring for sleep.

In certain embodiments, the step of administering an effective amount of the antitussive composition to the individual results in the individual receiving an effective amount of a sleep aid. In formulations including melatonin, this could be from about 0.1 mg to about 70 mg of melatonin. In related embodiments, the individual receives from about 1 mg to about 10 mg melatonin. In related embodiments, the individual receives from about 3 to about 5 mg melatonin.

In certain embodiments, the composition is administered to the individual perorally. To aid in peroral administration, a composition in forms advantageous for peroral administration may be used. For example, the compound to be administered may be in one of the following forms: an aqueous-based liquid, a syrup, a suspension, a spray, a lozenge, a chewable lozenge, a powder, a quick-dissolve powder, a quick-dissolve strip, a quick-dissolve tablet, a tablet, or a chewable tablet.

In one embodiment, the composition is initially in a powdered form, and prior to administration of the compound, the composition is dissolved in water or an aqueous-based liquid. In some of these, the composition is dissolved in cold, warm, lukewarm or hot water.

It will be obvious to those having skill in the art that many changes may be made to the details of the above-described embodiments without departing from the underlying principles of the invention. The scope of the present invention should, therefore, be determined only by the following claims.

1. An antitussive composition comprising honey and at least one sleep aid.

2. The antitussive composition of claim 1, wherein the at least one sleep aid is selected from the group consisting of 5-hydroxytryptophan (5-HTP), gamma-aminobutyric acid (GABA), glycine, L-theanine, L-tryptophan, melatonin, chamomile flower, lemon balm, passion flower, and valerian.

3. The antitussive composition of claim 1, further comprising an orally acceptable carrier, wherein the composition is substantially in one of the following forms: an aqueous-based liquid, a syrup, a suspension, a spray, a lozenge, a chewable lozenge, a powder, a quick-dissolve powder, a quick-dissolve strip, a quick-dissolve tablet, a tablet or a chewable tablet.

4. The antitussive composition of claim 3, wherein the composition is substantially in a powder form.

5. The antitussive composition of claim 4, wherein the composition comprises melatonin.

6. The antitussive composition of claim 5, wherein the composition comprises melatonin at a concentration from about 0.0007% (w/w) to about 0.5% (w/w).

7. The antitussive composition of claim 1, wherein the composition is in a dosage unit form.

8. The antitussive composition of claim 7, wherein each dosage unit comprises from about 0.1 mg to about 70 mg melatonin.

9. The antitussive composition of claim 1, wherein the honey is granular honey.

10. The antitussive composition of claim 1, wherein the honey is granular buckwheat honey.

11. The antitussive composition of claim 1, wherein the composition further comprises elderberry extract.

12. The antitussive composition of claim 1, wherein the composition further comprises grape extract.

13. The antitussive composition of claim 1, wherein the composition further comprises grape seed extract.

14. The antitussive composition of claim 1, wherein the composition further comprises grape skin extract.

15. The antitussive composition of claim 1, wherein the composition further comprises green tea extract.

16. The antitussive composition of claim 1, wherein the composition further comprises one or more added B-vitamins.

17. The antitussive composition of claim 1, wherein the composition further comprises added vitamin C.

18. The antitussive composition of claim 1, wherein the composition further comprises added vitamin D.

19. The antitussive composition of claim 1, wherein the composition further comprises added minerals.

20. The antitussive composition of claim 19, wherein the composition comprises added zinc.

21. A method of providing relief from coughing to an individual, comprising the step of administering to the individual an effective amount of an antitussive composition comprising honey and a sleep aid.

22. The method of claim 21, wherein the sleep aid is selected from the group consisting of 5-hydroxytryptophan (5-HTP), gamma-aminobutyric acid (GABA), glycine, L-theanine, L-tryptophan, melatonin, chamomile flower, lemon balm, passion flower, and valerian.

23. The method of claim 21, wherein the step of administering an effective amount of the antitussive composition to the individual results in the individual receiving from about 0.1 mg to about 70 mg of melatonin.

24. The method of claim 21, wherein the coughing is night coughing, and wherein the step of administering an effective amount of the antitussive composition to the individual is performed just prior to the individual retiring for sleep.

25. The method of claim 21, wherein the antitussive composition is administered perorally.

26. The method of claim 25, wherein the antitussive composition is in powder form, and wherein prior to the step of administering the antitussive composition, the antitussive composition is dissolved in an aqueous-based liquid.
